(19) United States
(12) Patent Application Publication
(10) Pub. No.: US 2021/0358622 A1
(43) Pub. Date: Nov. 18, 2021

(54) IMAGE ANALYSIS INCLUDING SYNTHETIC IMAGES

(71) Applicant: International Business Machines Corporation, Armonk, NY (US)

(72) Inventors: Sun Young Park, San Diego, CA (US); Dustin Michael Sargent, San Diego, CA (US); Arun Krishnan, Acton, MA (US)

(21) Appl. No.: 15/931,640

(22) Filed: May 14, 2020

(51) Int. Cl. G16H 50/20; G06T 7/00; G16H 30/40; G16H 10/60; G06T 7/38

(52) U.S. Cl. CPC G16H 50/20; G06T 7/0012; G16H 30/40; G06T 2207/20084; G06T 7/38; G06T 2207/30004; G06T 2207/20081; G16H 10/60

(57) ABSTRACT

Image sequence analysis by receiving a set of sequential images associated with a timeline, determining a gap according to the set of sequential images, generating a synthetic image associated with the gap according to the set of sequential images, and providing a new set of images including the synthetic image.

### BACKGROUND

The disclosure relates generally to computer aided detection (CAD) analysis of a temporal sequence of images. The disclosure relates particularly to CAD analysis of a sequence of medical images including synthetic images.

Computer Aided Detection analysis of images, including medical diagnostic images associated with conditions such as breast cancer, help radiologists review patient diagnostic imaging. The pattern recognition CAD systems decrease oversights and reduce false negatives arising from image analysis. CAD refers to machine learning, or artificial intelligence software systems which are trained to analyze images and to call attention to image patterns associated with suspicious features. The identified features are then subject to further review by a radiologist. CAD systems analyze single images as well as sequences of images taken over time, e.g., once a year, for a patient. Factors including patient hormone usage, menopause and patient ages may affect imaging conditions and the subsequent CAD analysis.

### SUMMARY

The following presents a summary to provide a basic understanding of one or more embodiments of the disclosure. This summary is not intended to identify key or critical elements or delineate any scope of the particular embodiments or any scope of the claims. Its sole purpose is to present concepts in a simplified form as a prelude to the more detailed description that is presented later. In one or more embodiments described herein, devices, systems, computer-implemented methods, apparatuses and/or computer program products enable synthetic image generation and image sequence analysis.

Aspects of the invention disclose methods, systems and computer readable media associated with image sequence analysis by receiving a set of sequential images associated with a timeline, determining a gap according to the set of sequential images, generating a synthetic image associated with the gap according to the set of sequential images, and providing a new set of images including the synthetic image.

### BRIEF DESCRIPTION OF THE DRAWINGS

Through the more detailed description of some embodiments of the present disclosure in the accompanying drawings, the above and other objects, features and advantages of the present disclosure will become more apparent, wherein the same reference generally refers to the same components in the embodiments of the present disclosure.

FIG. 1 provides a flowchart of operational training steps, according to an embodiment of the invention.

FIG. 2 provides a flowchart of image generator training steps, according to an embodiment of the invention.

FIG. 3 provides a flowchart of computer aided detection model training steps, according to an embodiment of the invention.

FIG. 4 provides a schematic illustration of a computing environment according to an embodiment of the invention.

FIG. 5 provides a flowchart depicting an operational sequence, according to an embodiment of the invention.

FIG. 6 depicts a cloud computing environment, according to an embodiment of the invention.

FIG. 7 depicts abstraction model layers, according to an embodiment of the invention.

### DETAILED DESCRIPTION

Some embodiments will be described in more detail with reference to the accompanying drawings, in which the embodiments of the present disclosure have been illustrated. However, the present disclosure can be implemented in various manners, and thus should not be construed to be limited to the embodiments disclosed herein.

In an embodiment, one or more components of the system can employ hardware and/or software to solve problems that are highly technical in nature (e.g., training machine learning systems, using machine learning systems to generate and analyze images, etc.). These solutions are not abstract and cannot be performed as a set of mental acts by a human due to the processing capabilities needed to facilitate Computer Aided Detection using generated images, for example. Further, some of the processes performed may be performed by a specialized computer for carrying out defined tasks related to memory operations. For example, a specialized computer can be employed to carry out tasks related to image generation and image sequence analysis, or the like.

Throughout the detailed description, reference is made to medical diagnostic image sequences and patient data, this is for purposes of description only and is not intended to limit the scope of the claimed invention.

Radiologists utilize computer aided detection (CAD) systems to assist in screening patient images for underlying conditions such as breast cancer. Comparing current imagery with prior images increases the effectiveness of the CAD screening tools. CAD systems benefit from an input of a series of sequential diagnostic images acquired at regular intervals. Such images tend to be associated with regular patient check-ups. Having the sequence of regularly spaced images enables the CAD system to more easily recognize patterns associated with suspicious features of the underlying condition. Patients do not always follow regular screening recommendations and complete sets of prior images at the proper timing intervals are not always available. Image analysis may be affected by one or more image factors, such as image quality, image view angle as well as image timing. Follow up images outside the typical screening imaging timeline acquired to address image quality, view angle or other image issues may be present in the set of patient images. Patient factors such as age, surgery history, hormone usage, and menopause may render some prior imagery irrelevant to a current CAD analysis. Disclosed systems and methods enable the proper grouping of available imagery into sets of relevant and irrelevant imagery, generate synthetic images needed for a proper analysis, and apply CAD screening to a sequence of uniformly spaced diagnostic images.

In an embodiment, CAD refers to computer aided diagnostics in addition to computer aided detection. In this embodiment, the CAD system receives a set of diagnostic images, including synthetic images, conducts feature detection analysis upon the current images according to the trained model and also conducts a diagnostic analysis according to a second model trained according to image sequence and diagnostic outcome data.

In an embodiment, the disclosed methods accept patient demographic and health history data as well as screening schedules and diagnostic guidelines appropriate to the relevant medical condition, in conjunction with medical diagnostic imagery. The trained machine learning systems of the disclosure separate the provided images into relevant and irrelevant categorizations according to the patient demographic, screening and health data. The method evaluates the relevant images to determine if there are gaps in the sequence of images according to the guidelines, patient risk factors and screening schedule—years for which no image is present among the sequence of relevant images. The method then generates synthetic images to fill the gaps. In an embodiment, the method generates the images using a generator network from a generative adversarial network (GAN), trained to generate images according to provided patient demographic and health data.

In an embodiment, the synthetic images are added to the provided images yielding a complete set of prior and current images for the CAD analysis. Each sequence of images will contain a variable number of images with the images having uniform time intervals between them. In this embodiment, the CAD analysis considers the current and prior images to identify suspicious features in the current image. The CAD analysis also considers the sequence of regularly spaced images in terms of diagnostic outcomes associated with the sequence.

In an embodiment, the disclosed method utilizes a number of different machine learning models. A first machine learning model receives a set of images together with image subject attribute data and parses the set of images into relevant and irrelevant groups. As an example, the model receives a sequence of mammogram images for a patient together with patient demographic and health data for the time of each image. Data including patient age, menopausal status, breast imaging reporting and database system (BI-RADS) score, CAD results, breast density, radiologist notes, hormone use data etc., are provided for each sequence image.

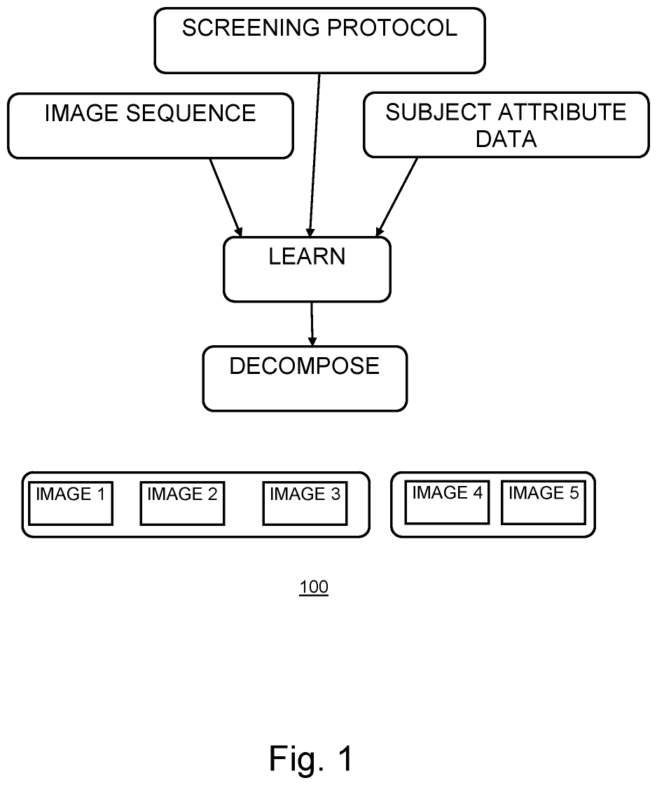

FIG. 1 provides a flowchart 100 of operational steps associated with training the first machine learning model. Training the model includes providing actual image sequences and training the model to properly separate the images according to the patient data associated with each image. For model training, complete image sequences are provided to the model. As shown in the figure, the machine learning model receives images, screening protocols including timing of screening imaging, and patient attribute data as described above. Through training the model learns to decompose the input image sequence into image groups relevant to the current image and irrelevant to the current image. As shown in the figure, the trained machine learning model decomposes new image sequences into groups relevant—images 4 and 5—and irrelevant—images 1, 2, and 3—to the current image.

In an embodiment, the method creates a feature vector for each image of a sequence. In this embodiment, the feature vector includes the patient demographic and medical history data associated with each image. During the training phase, the node weights of the model are adjusted according to the decomposition, or separation, of image sequences into relevant and irrelevant sets according to image similarities/dissimilarities. The model learns to associate the image groupings with changes in the feature vectors, learning to group images according to feature vectors as well as images similarities.

In an embodiment, training the first machine learning model includes supervised learning wherein complete sequences of images and accompanying patient demographic and medical data are input to the model. Each complete sequence of provided images is manually divided into the groups and labeled. The labeled data is then provided to the machine learning network, such as a recurrent neural network (RNN) or a hidden Markov machine (HMM). In this embodiment, the node weights of the model are adjusted using gradient back propagation against a loss function together with the data labels to achieve a set of node weights which correctly categorizes the training data as relevant or irrelevant. Validating the model requires the analysis of additional sets of labeled image sequences into groups of relevant or irrelevant images.

In an embodiment, unsupervised training of the first machine learning model includes providing unlabeled data to the machine learning model, again either an RNN, HMM, or similar machine learning architecture, and using clustering methods, k-means, graph cut, etc., to group similar images together and to adjust node weights such that the model learns to cluster the similar images into relevant and irrelevant groups. Models trained using unsupervised learning are validated using additional image sequences.

New image sequences processed and decomposed by the trained first machine learning model may include image gaps corresponding to time intervals where the patient did not have diagnostic imaging taken. In an embodiment, the method determines gaps according to the timing of the provided images and the screening protocol timing. Filling the gaps with synthetic images requires a second machine learning system for image generation. In an embodiment, a three-dimensional (3D) image generator creates the synthetic images required to fill image sequence gaps. In an embodiment, a generator network of a three-dimensional cycle-consistent generative adversarial network (3D CycleGAN) provides the synthetic images.

The 3DCycleGAN concurrently trains two generator networks and two discriminator networks. A first generator creates outputs for a second domain using images from a first domain as input. The second generator creates outputs for the first domain using images from the second domain as input. The first discriminator seeks to determine if the first domain images are real or generated and the second discriminator seeks to determine if the second domain images are real or generated. Cycle consistency for the 3D CycleGAN seeks a network state where the first generator processes an original input image from the first domain and produces an output image. That output image is passed to the second generator which accepts it as an input and produces an output image identical to the original first domain input image used by the first generator. Similarly, an output image from the second generator produced from an original second domain input image and then passed to the first generator should yield the original second domain image as the first generator output.

Disclosed embodiments utilize patient medical diagnostic images and accompanying patient demographic and health history information as an example data source for the methods. All disclosed methods presume that the relevant patients have opted-in, or otherwise consented, to providing access to patient images and information for use by the disclosed embodiments. In an embodiment, the 3D CycleGAN training includes taking incomplete as well as complete sequences of patient diagnostic images, together with accompanying patient demographic and health data, filling initial sequence gaps with blank images—all image pixels are “0”—and passing the stack of image to the first generator. The first generator alters the blank image attempting to generate a complete stack of sequenced images. The training method passes the generator output to a first discriminator. The first discriminator receives image stacks from the first generator (generated stacks) as well as complete image stacks (real stacks). Training proceeds in alternating epochs, alternating between training the discriminator and then the generator. In a first epoch, the weights of the discriminator are adjusted while those of the generator remain fixed. The discriminator adjusts weights using gradient back propagation to minimize a network loss function and accurately identify input stacks as real or generated. In a second epoch of training, the weights of the discriminator remain fixed and those of the generator are adjusted. The weights of the generator are adjusted using gradient back propagation beginning with the output layer of the discriminator and seeking to maximize a discriminator loss function for identifying real and generated inputs. Training epochs proceed alternating between the discriminator and generator until the GAN is well-trained. Initially, generated images are easily distinguished by the discriminator. With each training epoch, the generator improves its ability to generate a real looking image. For a well-trained GAN, the discriminator has a success rate of 50% in identifying real and generated inputs. (The generator has a 50% chance of fooling the discriminator as the generated images are indistinguishable from the real images).

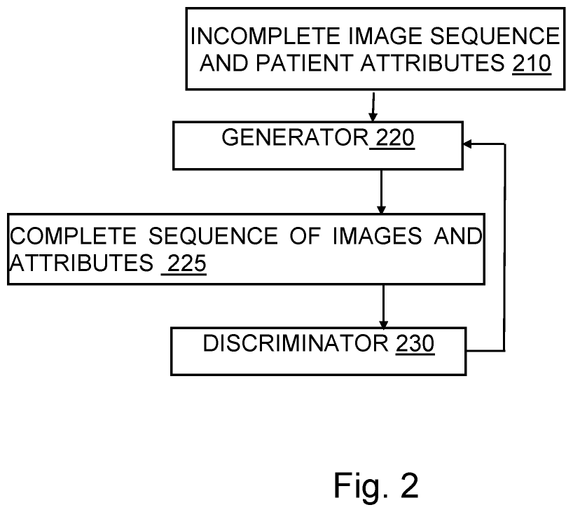

FIG. 2 illustrates the use of the CycleGAN. As shown in the figure, the trained CycleGAN generator 220 receives an incomplete image sequence together with image subject attribute data 210 (patient demographic and health history data). The generator 220 creates synthetic images to fill gaps in the input sequence according to the images preceding and following the gap in the sequence of images. The generator 220 creates realistic looking images which fit in the gaps of the original incomplete image sequence. In an embodiment, the generator also generates synthetic image subject attribute data—patient demographic and medical data—associated with the synthetic images. In this embodiment, the method labels the synthetic patient data to clearly identify it as generated and not real patient data. The generator 220 of the method passes the complete sequence of images 225 along for CAD review and analysis. In an embodiment, the generator 220 passes the completed sequence 225 to the discriminator 230 as well for review and to refine the node weights of the discriminator 230, and the generator 220.

In this embodiment, for cycle consistency, a second generator receives complete stacks as input, generates gaps in the complete stacks and passes the generated stacks to a second discriminator seeking to identify real and generated stacks having sequence gaps. Training of these second generator and discriminator networks proceeds with alternating training epochs as described above.

In an embodiment, the method passes the completed sequences to an external CAD system for review and analysis. The completed sequence includes one or more synthetic images and provides a sequence of images having uniform spacing in terms of the time between images. In this embodiment, the external CAD system provides analysis of the current image in view of the completed sequence to augment the review of the current image by a radiologist.

In an embodiment, the completed sequence including one or more synthetic images passes to an internal CAD system for review. In this embodiment, the internal CAD system includes a third machine learning model, including a sequential deep learning model such as a Long-Short Term Memory (LSTM), RNN, or temporal convolution network (TCN) architecture adapted to analyze patterns in a temporal sequence of data such as the (now complete) temporal sequence of imaging data with uniform timing.

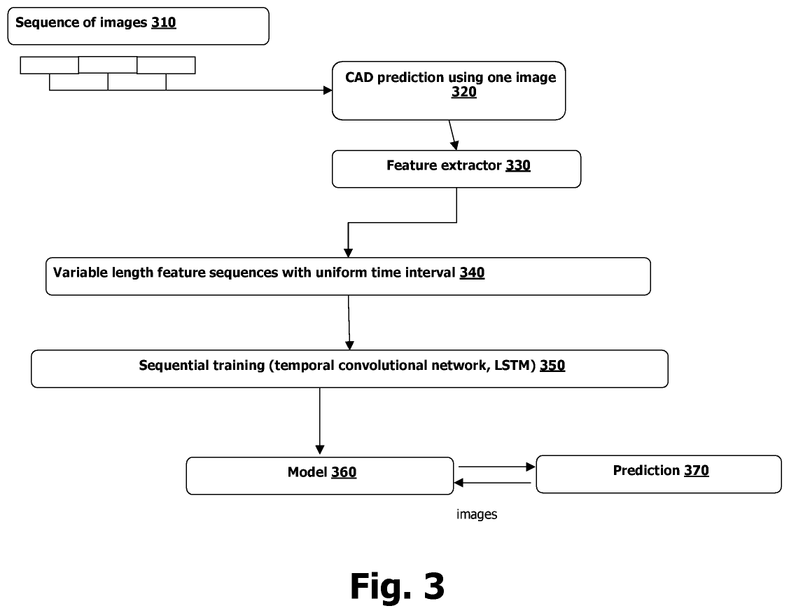

FIG. 3 illustrates steps associated with training the third machine learning model according to an embodiment of the invention. As shown in the figure, training steps include manually labeling suspicious features of each image in each image sequence of a training data set of image sequences having uniform time intervals, and providing the set of labeled image sequences at block 310; running each labeled image against a computer aided detection algorithm at block 320, to obtain a 1 dimensional feature vector at block 330, for each image associated with the CAD algorithm output; and associating the 1D feature vector with the labelling of the data. The training data set sequences with uniform time intervals, and now including manually applied labels and CAD algorithm variable feature length outputs from block 340, are used to train the sequential deep learning model to recognize diagnostic relevant patterns developing across the sequence of images at block 350. The third machine learning (such as LSTM) model 360 learns to provide feature recognition and diagnostic outputs from the input image sequences, block 370.

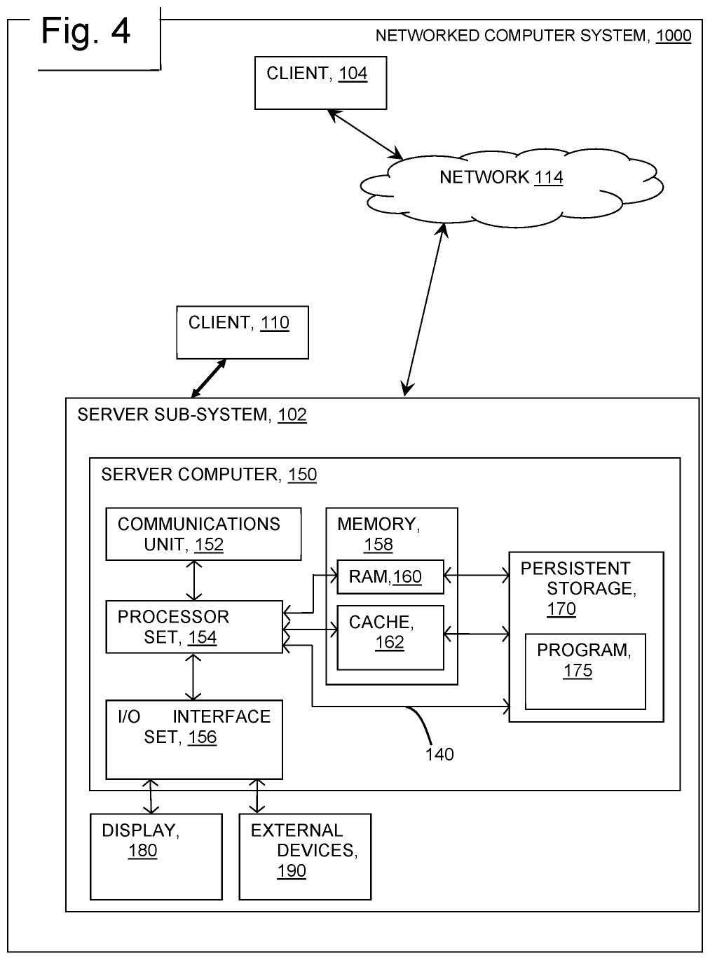

FIG. 4 provides a schematic illustration of exemplary network resources associated with practicing the disclosed inventions. The inventions may be practiced in the processors of any of the disclosed elements which process an instruction stream. As shown in the figure, a networked Client device 110 connects wirelessly to server sub-system 102. Client device 104 connects wirelessly to server sub-system 102 via network 114. Client devices 104 and 110 comprise application program (not shown) together with sufficient computing resource (processor, memory, network communications hardware) to execute the program. As shown in FIG. 4, server sub-system 102 comprises a server computer 150. FIG. 4 depicts a block diagram of components of server computer 150 within a networked computer system 1000, in accordance with an embodiment of the present invention. It should be appreciated that FIG. 4 provides only an illustration of one implementation and does not imply any limitations with regard to the environments in which different embodiments can be implemented. Many modifications to the depicted environment can be made.

Server computer 150 can include processor(s) 154, memory 158, persistent storage 170, communications unit 152, input/output (I/O) interface(s) 156 and communications fabric 140. Communications fabric 140 provides communications between cache 162, memory 158, persistent storage 170, communications unit 152, and input/output (I/O) interface(s) 156. Communications fabric 140 can be implemented with any architecture designed for passing data and/or control information between processors (such as microprocessors, communications and network processors, etc.), system memory, peripheral devices, and any other hardware components within a system. For example, communications fabric 140 can be implemented with one or more buses.

Memory 158 and persistent storage 170 are computer readable storage media. In this embodiment, memory 158 includes random access memory (RAM) 160. In general, memory 158 can include any suitable volatile or non-volatile computer readable storage media. Cache 162 is a fast memory that enhances the performance of processor(s) 154 by holding recently accessed data, and data near recently accessed data, from memory 158.

Program instructions and data used to practice embodiments of the present invention, e.g., the machine learning program 175, are stored in persistent storage 170 for execution and/or access by one or more of the respective processor(s) 154 of server computer 150 via cache 162. In this embodiment, persistent storage 170 includes a magnetic hard disk drive. Alternatively, or in addition to a magnetic hard disk drive, persistent storage 170 can include a solid-state hard drive, a semiconductor storage device, a read-only memory (ROM), an erasable programmable read-only memory (EPROM), a flash memory, or any other computer readable storage media that is capable of storing program instructions or digital information.

The media used by persistent storage 170 may also be removable. For example, a removable hard drive may be used for persistent storage 170. Other examples include optical and magnetic disks, thumb drives, and smart cards that are inserted into a drive for transfer onto another computer readable storage medium that is also part of persistent storage 170.

Communications unit 152, in these examples, provides for communications with other data processing systems or devices, including resources of client computing devices 104, and 110. In these examples, communications unit 152 includes one or more network interface cards. Communications unit 152 may provide communications through the use of either or both physical and wireless communications links. Software distribution programs, and other programs and data used for implementation of the present invention, may be downloaded to persistent storage 170 of server computer 150 through communications unit 152.

I/O interface(s) 156 allows for input and output of data with other devices that may be connected to server computer 150. For example, I/O interface(s) 156 may provide a connection to external device(s) 190 such as a keyboard, a keypad, a touch screen, a microphone, a digital camera, and/or some other suitable input device. External device(s) 190 can also include portable computer readable storage media such as, for example, thumb drives, portable optical or magnetic disks, and memory cards. Software and data used to practice embodiments of the present invention, e.g., machine learning program 175 on server computer 150, can be stored on such portable computer readable storage media and can be loaded onto persistent storage 170 via I/O interface(s) 156. I/O interface(s) 156 also connect to a display 180.

Display 180 provides a mechanism to display data to a user and may be, for example, a computer monitor. Display 180 can also function as a touch screen, such as a display of a tablet computer.

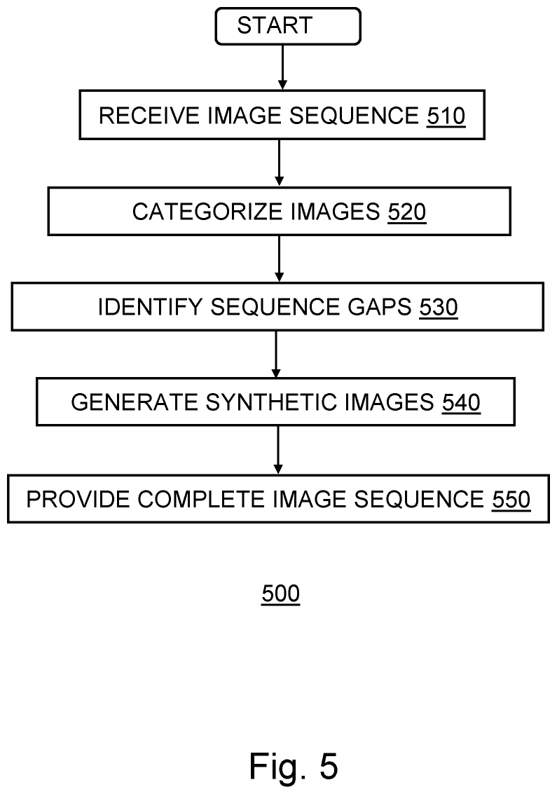

FIG. 5 provides a flowchart 500, illustrating exemplary activities associated with the practice of the disclosure. After program start, at block 510, the image analysis program 175 receives diagnostic image sequences including diagnostic images and image subject attribute data (patient demographic and health data). The image sequence includes a current image. The images of the sequence are time stamped and generally associated with a subject timeline-patient care timeline. The relative timing of the sequence images may be irregular or may be regular with some gaps—years where no imaging was conducted or with missing data. At block 520 a first machine learning model categorizes the images of the received sequence as either relevant to the current image analysis or irrelevant to the current analysis. The first machine learning model categorizes the images according to the patient demographic and health data associated with the images.

At block 530, the method of image analysis program 175 analyzes the categorized image groups to identify gaps in the image sequence timing of the grouping of relevant images.

At block 540, a second machine learning generator model creates synthetic images to fill any gaps identified in block 530. In an embodiment, the generator is derived from a CycleGAN machine learning model trained using both incomplete and complete sets of patient diagnostic imaging sequences. In an embodiment, the generator receives the incomplete sequence of images and patient data as an input and completes the sequence by creating realistic synthetic images.

At block 550, the method of image analysis program 175 provides the now complete medical diagnostic image sequence or stack to a user for further analysis using a CAD system and to augment manual review of the current image by a radiologist.

In an embodiment, the method passes the now complete set of images to a CAD module including a third machine learning model trained to analyze temporal sequence data, which identifies suspicious feature in the current image as well as providing a diagnosis according to the training of the third machine learning model. In this embodiment, the third machine learning model provide the identified suspicious features and diagnosis as outputs.

In an embodiment, the method utilizes cloud and/or edge cloud computing resources for the training and deployment of the multiple machine learning models utilized in generating images and analyzing sequences of images. Utilizing the cloud and/or edge cloud resources enables the method to complete the computationally intensive machine learning model training steps more efficiently and in a timelier manner.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

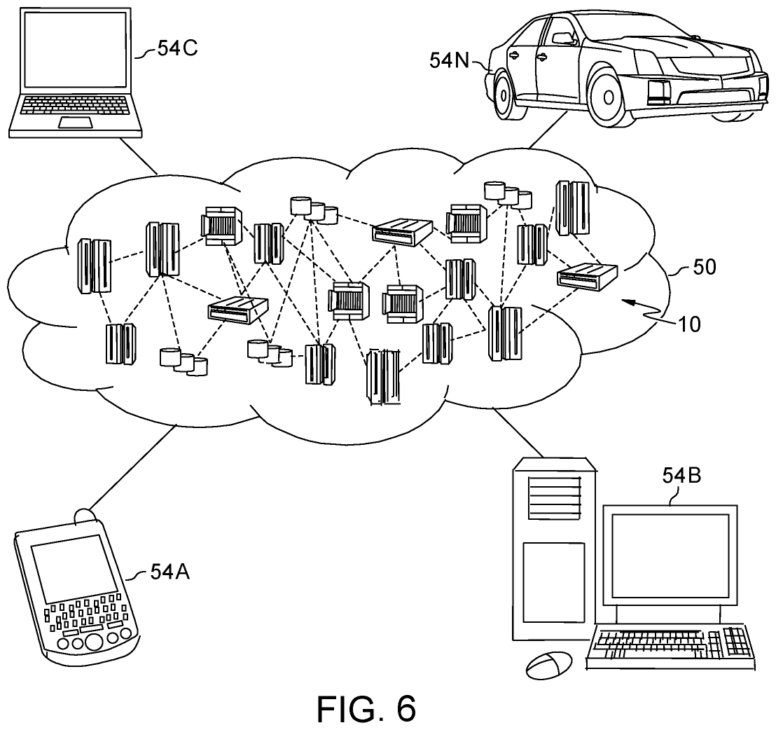

Referring now to FIG. 6, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 6 are intended to be illustrative only and that computing nodes 10 and cloud computing environment 50 can communicate with any type of computerized device over any type of network and/or network addressable connection (e.g., using a web browser).

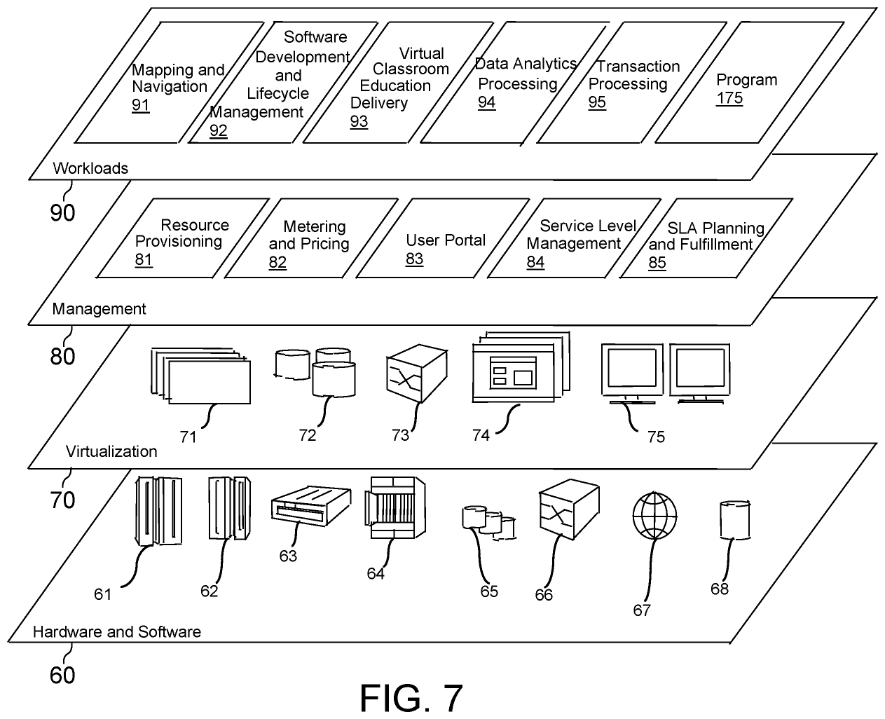

Referring now to FIG. 7, a set of functional abstraction layers provided by cloud computing environment 50 (FIG. 6) is shown. It should be understood in advance that the components, layers, and functions shown in FIG. 7 are intended to be illustrative only and embodiments of the invention are not limited thereto. As depicted, the following layers and corresponding functions are provided.

Hardware and software layer 60 includes hardware and software components. Examples of hardware components include: mainframes 61; RISC (Reduced Instruction Set Computer) architecture-based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and machine learning program 175.

The present invention may be a system, a method, and/or a computer program product at any possible technical detail level of integration. The invention may be beneficially practiced in any system, single or parallel, which processes an instruction stream. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

References in the specification to “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the invention. The terminology used herein was chosen to best explain the principles of the embodiment, the practical application or technical improvement over technologies found in the marketplace, or to enable others of ordinary skill in the art to understand the embodiments disclosed herein.

What is claimed is:

1. A computer implemented method for image sequence analysis, the method comprising:
receiving a set of sequential images associated with a timeline;
determining a gap in the set of sequential images;
generating a synthetic image associated with the gap according to the set of sequential images; and
providing a new set of images including the synthetic image.

2. The computer implemented method according to claim 1, wherein the synthetic image is generated according to images in the set of sequential images preceding and following the gap.

3. The computer implemented method according to claim 1, further comprising generating synthetic subject attributes associated with the synthetic image.

4. The computer implemented method according to claim 3, further comprising:
categorizing the set of sequential images according to image subject attributes;
analyzing the set of images including the synthetic image, and the synthetic subject attributes using a computer aided detection model, and
providing a computer aided detection model output.

5. The computer implemented method according to claim 1, wherein the set of sequential images comprises medical diagnostic images.

6. The computer implemented method according to claim 5, wherein determining the gap in the set of sequential images is based, at least in part, upon a medical condition.

7. The computer implemented method according to claim 1, wherein the set of sequential images comprises medical diagnostic images associated with a medical condition, the image subject attributes comprise patient demographic and/or medical history data, and determining the gap in the set of images comprises determining a gap according to the medical condition and patient demographic and/or medical history data.

8. A computer program product for image sequence analysis, the computer program product comprising one or more computer readable storage devices and program instructions collectively stored on the one or more computer readable storage devices, the stored program instructions comprising:
program instructions to receive a set of sequential images associated with a timeline;
program instructions to determine a gap in the set of sequential images;
program instructions to generate a synthetic image associated with the gap according to the set of sequential images; and
program instructions to provide a new set of images including the synthetic image.

9. The computer program product according to claim 8, wherein the synthetic image is generated according to images preceding and following the gap.

10. The computer program product according to claim 8, the stored program instructions further comprising program instructions to generate synthetic image subject attributes.

11. The computer program product according to claim 10, the stored program instructions further comprising:
program instructions to categorize the set of sequential images according to image subject attributes;
program instructions to analyze the set of images including the synthetic image and the synthetic subject attributes, using a computer aided detection model; and
providing a computer aided detection model output.

12. The computer program product according to claim 8, wherein the set of sequential images comprises medical diagnostic images.

13. The computer program product according to claim 12, wherein determining the gap in the set of sequential images is based, at least in part, upon a medical condition.

14. The computer program product according to claim 8, wherein the set of sequential images comprises medical diagnostic images associated with a medical condition, the image subject attributes comprise patient demographic and/or medical history data, and the program instructions to determine the gap comprise program instructions to determine a gap according to the medical condition and patient demographic and/or medical history data.

15. A computer system for image sequence analysis, the computer system comprising:
one or more computer processors;
one or more computer readable storage devices; and
stored program instructions on the one or more computer readable storage devices for execution by the one or more computer processors, the stored program instructions comprising:
program instructions to receive a set of sequential images associated with a timeline;
program instructions to determine a gap in the set of sequential images;
program instructions to generate a synthetic image associated with the gap according to the set of sequential images; and
program instructions to provide a new set of images including the synthetic image.

16. The computer system according to claim 15, wherein the synthetic image is generated according to images preceding and following the gap.

17. The computer system according to claim 15, the stored program instructions further comprising program instructions to generate synthetic image subject attributes.

18. The computer system according to claim 17, the stored program instructions further comprising:
program instructions to categorize the set of sequential images according to image subject attributes;
program instructions to analyze the set of images including the synthetic image and the synthetic subject attributes, using a computer aided detection model; and
providing a computer aided detection model output.

19. The computer system according to claim 18, wherein the set of sequential images comprises medical diagnostic images.

20. The computer system according to claim 15, wherein the set of sequential images comprises medical diagnostic images associated with a medical condition, the image subject attributes comprises patient demographic and/or medical history data, and the program instructions to determine the gap comprise program instructions to determine a gap according to the medical condition and patient demographic data.
